(19) United States
(12) Plant Patent Application Publication
(10) Pub. No.: US 2025/0386754 P1
(43) Pub. Date: Dec. 18, 2025

(54) Heliopsis helianthoides var. scabra Plant named 'BullHeliopsisscabra 01'

(50) ;

(71) Applicant: Hartwig Bull, Gönnebek (DE)

(72) Inventor: Hartwig Bull, Gönnebek (DE)

(21) Appl. No.: 19/235,276

(22) Filed: Jun. 11, 2025

Related U.S. Application Data
(60) Provisional application No. 63/731,836, filed on Jun. 12, 2024.

(51) Int. Cl. A01H 6/14; A01H 5/02

(52) U.S. Cl. CPC A01H 6/14; A01H 5/02
USPC PLT/437

(57) ABSTRACT

A new and distinct variety of Heliopsis plant having a compact growth habit and yellow-orange blooms.

### CROSS REFERENCE TO RELATED APPLICATIONS

This application claims the benefit of priority from U.S. Provisional Patent Application No. 63/731,836, filed Jun. 12, 2024, which is incorporated herein by reference in its entirety.

Botanical classification: Heliopsis helianthoides var. scabra.

Varietal denomination: ‘BullHeliopsisscabra 01’.

### BACKGROUND OF THE INVENTION

The present invention relates to a new and distinct cultivar of Heliopsis plant, known by the varietal name ‘BullHeliopsisscabra 01’. The new variety was discovered in August of 2020 in Gönnebek, Germany as the result of a planned breeding program with the purposes of providing compact Heliposis plants with different flower colors, good growth habit, improved uniformity, good branching, good propagation rates, and that perform well. The new variety is the result of a cross between an unpatented Heliopsis variety having an internal breeder's reference of “BulHs 2018-03” (female parent) and an unpatented Heliopsos variety having an internal breeder's reference of “BulHs-2018-11” (male parent) from the breeder's own collection. The new variety was first asexually reproduced via in-vitro tissue culture cuttings in Gönnebek, Germany in September of 2020. The new variety is similar to its parental varieties in botanical classification, but exhibits more compactness, a greater number of flowers, specifically yellow-orange flowers, with good self-branching, more branches, nicer habit, and dark-green small leaves than the parental varieties.

When ‘BullHeliopsisscabra 01’ is compared to Heliopsis variety named ‘Hohlspiegel’ (unpatented), ‘BullHeliopsisscabra 01’ is similar to ‘Hohlspiel’ in botanical classification and the presence of orange-yellow flowers, but ‘BullHeliopsisscabra 01’ exhibits greater compactness, a greater branching habit that is very even, a greater number of flowers, including pure orange flowers, and nicer, cleaner leaves, that distinguish it from ‘Hohlspiegel’. Further, the following characteristics distinguish ‘BullHeliopsisscabra 01’ when generally compared to other Heliopsis varieties known to the breeder:
Much more compact;
Good self-branching habit;
A great number of yellow-orange flowers;
Small leaves;
Very uniform growth;
Nice dark green leaves;
Fast crop time;
Long-lasting flowers; and
Good hardiness as a perennial.

The new variety has been trial and field tested and has been found to retain its distinctive characteristics and remain true to type through successive asexual propagations. The present invention has not been evaluated under all possible environmental conditions. The phenotype may vary with variations in environment without a change in the genotype of the plant.

### DESCRIPTION OF THE DRAWINGS

The accompanying photographic drawings illustrate the new variety at 20 weeks of age, with the color being as nearly true as is possible with color illustrations of this type:

FIG. 1 shows an entire plant of the new variety.

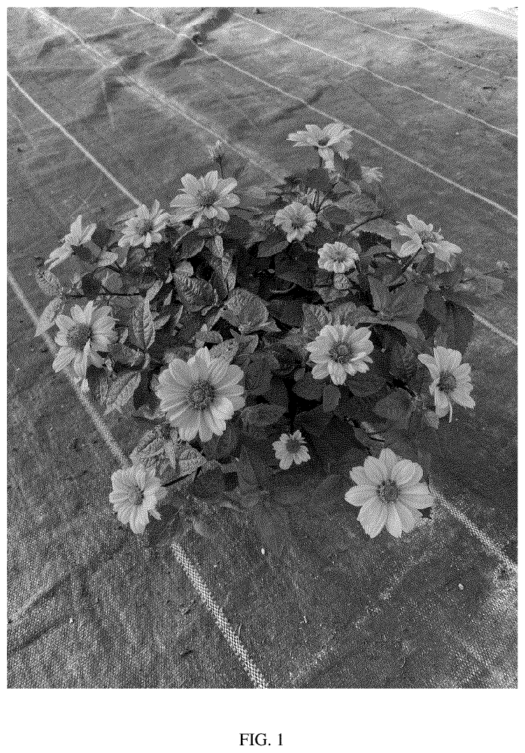

### DESCRIPTION OF THE PLANT

The following detailed description sets forth the characteristics of the new variety as the result of asexual reproductions performed via in vitro tissue culture cuttings carried out in Gönnebek, Germany. Plants of the new variety were grown outdoors in 15 cm (1.5 liter) pots under normal field production conditions (2 pinches), and the color readings and measurements were observed outdoors under natural light on 20-week-old plants in Gönnebek, Germany. Color references are primarily to The 2015 R.H.S. Colour Chart of The Royal Horticultural Society of London, Sixth Edition, except where terms of ordinary significance are used.

### Plant

Time to initiate roots: About 10 days at about 19-20° C.
Time to develop roots: About 40 days at about 18° C.
Time to produce a finished flowering plant from a rooted cutting: About 10-12 weeks in a 15 cm container.
Rooting habit and description: Lots of roots, free branching nice healthy-looking root system.
Plant height: 30.0-40.0 cm.
Plant width: 25.0-30.0 cm.
Vigor: Compact, upright.
Branching habit: Lot of branches, upright, shows a medium compact plant.
Lateral branches:
   Number per plant.—15-20.
   Length.—15.0-35.0 cm.
   Diameter.—3.0-6.0 mm.
   Internode length.—1.0-5.0 cm.
   Angle.—Upright, outward.
   Strength.—Strong.
   Texture.—Pubescent.
   Color.—Gray-brown (close to N199A).
Foliage:
   Arrangement.—Single.
   Leaf.—Acuminate. Length: 2.5-10.0 cm. Width: 1.0-5.0 cm. Shape: Acuminate. Apex: Acute. Base: Rounded to Acute. Texture: Upper surface: Rough. Lower surface: Rough. Aspect: Straight to curved. Margin: Serrate. Color: Young leaves: Upper surface: Green (close to NN137B). Lower surface: Green (close to 138B). Mature leaves: Upper surface: Green (close to NN137B). Lower surface: Green close to 138 B). Petiole: none. Length: 1-3 cm. Diameter: 2-3 mm. Texture: Strong. Color: Gray-brown (close to N199A). Veins: Venation type: cross venulate. Color: Upper surface: Gray-brown (close to N199A). Lower surface: Gray Brown (N199A) to green (close to N138B).

### Inflorescence

Bud:
   Appearance.—Elliptic sharped ray florets and tubular disk floret.
   Diameter.—1.5-3.0 cm.
   Length.—1.0-1.6 cm.
   Color.—Green (close to N147C).
   Natural flowering season.—July-September in Gönnebek, Germany, beginning approximately 10-12 weeks after planting.
Inflorescence form and disc/ray arrangement description: Single flower.
Average number of inflorescences per plant: 20-30.
Disc and ray floret arrangement: Single composite flower.
Number of inflorescences per branch: 1-3.
Fragrance: None.
Inflorescence:
   Diameter.—4.0-10.0 cm.
   Height (depth).—2.0-4.5 cm.
   Diameter of disc.—1.5-4.0 cm.
   Receptacle height.—1.0 cm.
   Receptacle diameter.—1.5-4.0 cm.
Ray florets:
   Number per inflorescence.—16-24.
   Arrangement.—In two or three whirls.
   Length.—1.5-3.5 cm.
   Width.—0.9-1.5 cm.
   Shape.—Elliptical.
   Apex.—Obtuse.
   Base.—Acute.
   Margin.—Entire.
   Texture.—Upper surface: Smooth, glabrous. Lower surface: Rough.
   Color.—When opening: Upper surface: Orange (close to N30A). Lower surface: Orange (close to 35A). At maturity: Upper surface: Orange (close to N30A). Lower surface: Orange (close to 35A). Ground color description: Orange.
   Venation.—Appearance: Color: Upper surface: Same color as florets. Lower surface: Same color as florets.
Disc florets:
   Number per inflorescence.—Numerous disk florets.
   Arrangement.—In center of receptacle.
   Length.—8.0-15.0 mm.
   Width.—1.0-2.0 mm.
   Shape.—Tubular.
   Apex.—Sharp.
   Color.—Immature: Orange (close up to 23A). At maturity: Dark Orange (close up to 25D).
Phyllaries:
   Number per inflorescence.—12-18.
   Arrangement.—In 2 whirls.
   Length.—0.3-0.7 cm.
   Width.—4.0-6.0 mm.
   Shape.—Ovate.
   Apex.—Elongate, oval.
   Base.—Fused.
   Margin.—Entire.
   Texture.—Upper surface: Rough. Lower surface: Rough.
   Color.—Immature: Upper surface: Green (close to NN 138B). Lower surface: Green (close to NN138B). At maturity: Upper surface: Green (close to NN 138B). Lower surface: Green (close to NN 138B).

### Reproductive Organs

Androecium:
   Presence.—On disk florets.
   Number (per floret).—One.
   Filament length.—1.0-2.0 mm.
   Filament color.—Orange (close to 23A).
   Anther.—Shape: Oval. Length: 1.0 mm. Color: Orange (close to 23B).
   Pollen.—Color: Yellow (close to 15B). Amount: Lots of Quantity.
Gynoecium:
   Presence.—In Disk and Ray florets.
   Pistil length.—1-2 mm.
   Stigma.—Shape: Two parted. Color: Brown (close to 23A).
   Style.—Length: 1.0-2.0 mm. Color: orange (close to 23A).
Description of seeds/fruits (if observed): 1.0-2.0 mm.

I claim:

1. A new and distinct variety of Heliopsis plant named ‘BullHeliopsisscabra 01’, as is herein illustrated and described.
